(19) United States
(12) Patent Application Publication
(10) Pub. No.: US 2002/0183845 A1
(43) Pub. Date: Dec. 5, 2002

(54) Multi-perforated non-planar device for anchoring cartilage implants and high-gradient interfaces

(76) Inventor: Kevin A. Mansmann, Paoli, PA (US)

Correspondence Address: Patrick D. Kelly, 11939 Manchester #403, St. Louis, MD, 63131, US

(21) Appl. No.: 10/011,933

(22) Filed: Nov. 30, 2001

Related U.S. Application Data
(60) Provisional application No. 60/250,091, filed on Nov. 30, 2000.

(51) Int. Cl. A61F 2/08

(52) U.S. Cl. 623/13.11; 623/14.12; 623/18.11

(57) ABSTRACT

This invention relates to a surgical implant which contains a relatively soft component, such as a hydrogel or gel-like component, that is affixed to a harder and stiffer material. One such implant is designed to replace a segment of damaged cartilage in a knee, hip, shoulder, or other joint. Instead of attempting to bond a gel-like material to a hard surface with a relatively flat interface, this improved device provides a “multi-perforated non-planar” (MP/NP) interface between the two types of material. This type of MP/NP interface can provide a stronger and more durable gel structure, and it can enable the water molecules in the gel component to disseminate and distribute compressive and shear loads in a more even manner, reducing the risk of tearing or other damage. As one example, this type of MP/NP interface can resemble a moderately thin layer that has been molded into a “waffle” surface, and provided with holes passing through various facets of the material, oriented in different directions. In one embodiment, this type of implant can provide a wet hydrogel surface on the articulating side, nestled within a supporting plastic shell that can be securely anchored to a prepared bone surface. In an alternate embodiment, the MP/NP layer can be bonded to a porous anchoring layer made of a biocompatible material that will promote tissue ingrowth into the anchoring layer.

### RELATED APPLICATION

This application claims priority, under 35 USC 119(e), based on provisional patent application 60/250,091, filed on Nov. 30, 2000 by the same inventor herein.

### FIELD OF THE INVENTION

This invention is in the field of surgical implants, such as for replacing damaged cartilage in a knee, hip, or other joint.

### BACKGROUND OF THE INVENTION

In various settings, it is necessary to create a strong, secure, and durable interface between a hard material (such as a hard plastic shell, or a hard bone surface that has been surgically prepared), and a much softer and weaker material, such as a hydrogel or other gel-like component that will be installed within the shell, or mounted on top of a bone surface.

When this type of interface is required in a device that will be subject to high compressive and/or shear forces, a simple flat interface between the soft gel, and the hard supporting or anchoring layer, may not be adequate to reinforce the boundary between the hard supporting material, and the soft material that rests on the hard support. Even if the chemical bonding at a flat interface between the two different types of material is strong, the gel portion of the device can still be damaged or destroyed relatively easily, if shear forces drive the shell or anchoring layer in one direction while the gel is being pushed in a different direction. Even if the shear forces are relatively low, they will effectively focus and concentrate their destructive power on the interface between the two types of material. If that interface is simply flat, it has minimal surface area, and it cannot distribute the load across a larger area to help reduce the tearing or shearing forces that will be generated at any particular point within the interface.

In addition, if an interface between a hard support and a gel-type layer is relatively flat and planar, rather than multi-directional, the gel is not given a fair opportunity to use its internal version of fluid flow to help it redistribute the load it must withstand. In a typical gel, water molecules are trapped within a three-dimensional network, made of long fibers or polymeric strands which are crosslinked to each other in ways that form a three-dimensional matrix; in a naturally-occurring gel such as cartilage, the fibers are made of collagen (a fibrous protein) and “proteoglycan” filaments. Within that fibrous matrix, none of the water molecules are attached to any particular strand. Therefore, water molecules can move about inside the gel, in a manner which is partly free and partly constrained.

In some situations, this might allow a gel material to resist a compressive or shear force by simply readjusting the water molecules within the gel, in a manner which allows the water to exert straight-on pressure against any fixed surface that is positioned in a direction that will allow it to press back against an imposed pressure. However, if a gel is bonded to a flat surface by nothing more than a simple flat interface, there are no such intervening or intruding surfaces that the water molecules can exert pressure against, to help them redistribute a load more evenly.

Accordingly, an improved device and method are disclosed herein, for bonding a pliable gel-type material to a more rigid supporting structure. The initial goal of this improved device is to provide an improved type of surgical implant, for replacing segments of damaged cartilage, in joints such as a knee, hip, shoulder, wrist, ankle, or finger. However, this same type of approach is likely to prove useful in various other types of surgical procedures.

In addition, this approach to creating a stronger and more durable and secure interface between a hard surface and a softer material is also likely to be useful in various non-surgical settings. As one example, various types of padding and/or protective devices used by athletes (such as boots used by skiers), various types of prosthetics, and various devices often referred to as “orthotic” appliances, might be significantly improved if a gel-type pad or other advanced padding system (as distinct from a conventional foam pad) could be secured to a shell or other anchoring or enclosing surface using an interface structure as disclosed herein.

Accordingly, one object of this invention is to provide an improved type of surgical implant which has a relatively strong and potentially stiff membrane or shell on one exposed side, for anchoring to a prepared bone surface or similar internal surface, and a substantially softer material (such as a hydrogel or gel-like material on the other side), wherein the two different materials are bonded to each other in a manner which provides improved strength and durability.

Another object of this invention is to disclose an improved interface layer for surgical implants designed to replace damaged cartilage in mammalian joints, which will provide superior load-distributing characteristics when a gel or gel-like component that emulates cartilage is secured to the improved interface layer.

Another object of this invention is to disclose an improved type of interface which can be provided in surgical implants that have two different materials bonded to each other, when one material is relatively stiff and strong, and the other material is substantially softer and more pliable.

Another object of this invention is to disclose an improved type of surgical implant for replacing a damaged segment of cartilage in a joint such as a knee, hip, or shoulder.

These and other objects of the invention will become more apparent through the following summary, drawings, and description of the preferred embodiments.

### SUMMARY OF THE INVENTION

This invention relates to a surgical implant which contains a relatively soft component, such as a hydrogel or gel-like component, that is affixed to a supporting, anchoring, or similar component made of a harder and stiffer material. One such implant is designed to replace a segment of damaged cartilage in a knee, hip, shoulder, or other joint. Instead of attempting to bond a gel-like material to a supporting or anchoring structure by means of a relatively flat interface, this improved device provides a “multi-perforated non-planar” (MP/NP) interface between the two types of material. This type of MP/NP interface can provide a stronger and more durable gel structure, and it can enable the water molecules in the gel component to disseminate and distribute compressive and shear loads in a more even manner, reducing the risk of tearing or other damage. As one example, this type of MP/NP interface can resemble a moderately thin layer that has been molded into a “waffle” surface, and provided with holes passing through various facets of the material, oriented in different directions. In one preferred embodiment, this type of cartilage replacement implant can provide a wet hydrogel surface on the articulating side, nestled within a supporting plastic shell that can be securely anchored to a prepared bone surface. In an alternate preferred embodiment, the MP/NP layer can be bonded to a porous anchoring layer made of a biocompatible material that will promote tissue ingrowth into the anchoring layer.

### BRIEF DESCRIPTION OF THE DRAWINGS

FIG. 1 depicts a multi-perforated non-planar (MP/NP) interface layer, with numerous “riser bumps” having five orifices in each bump, sized for an implant designed for replacing a segment of cartilage on a tibial plateau.

FIG. 2 depicts a close-up view of a small segment of an MP/NP interface layer, resting on a flat surface of an anchoring shell, where one of the “riser bumps” is shown in a cutaway manner.

FIG. 3 depicts a multi-layered implant in a cut-away side view, showing a hydrogel layer on the upper (articulating) surface, an MP/NP interface layer in the middle, and a porous anchoring layer that will promote tissue ingrowth after the implant is anchored to a prepared bone surface.

### DESCRIPTION OF THE PREFERRED EMBODIMENTS

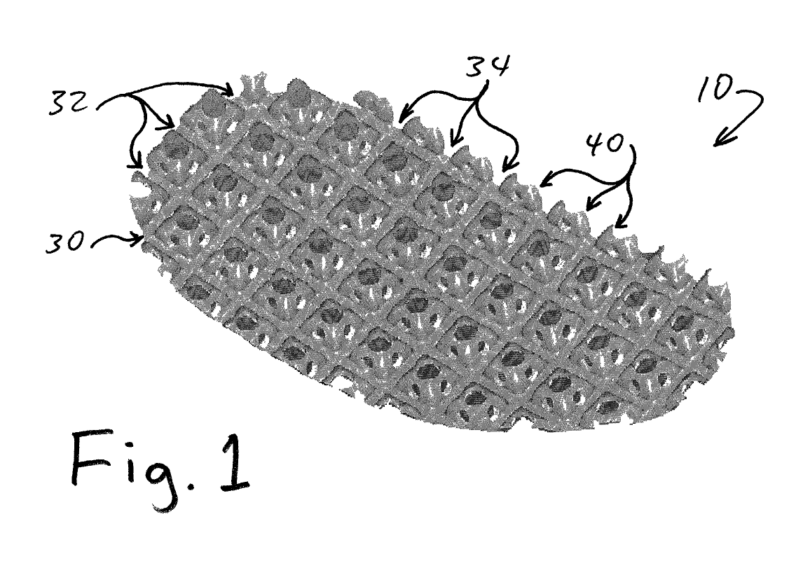

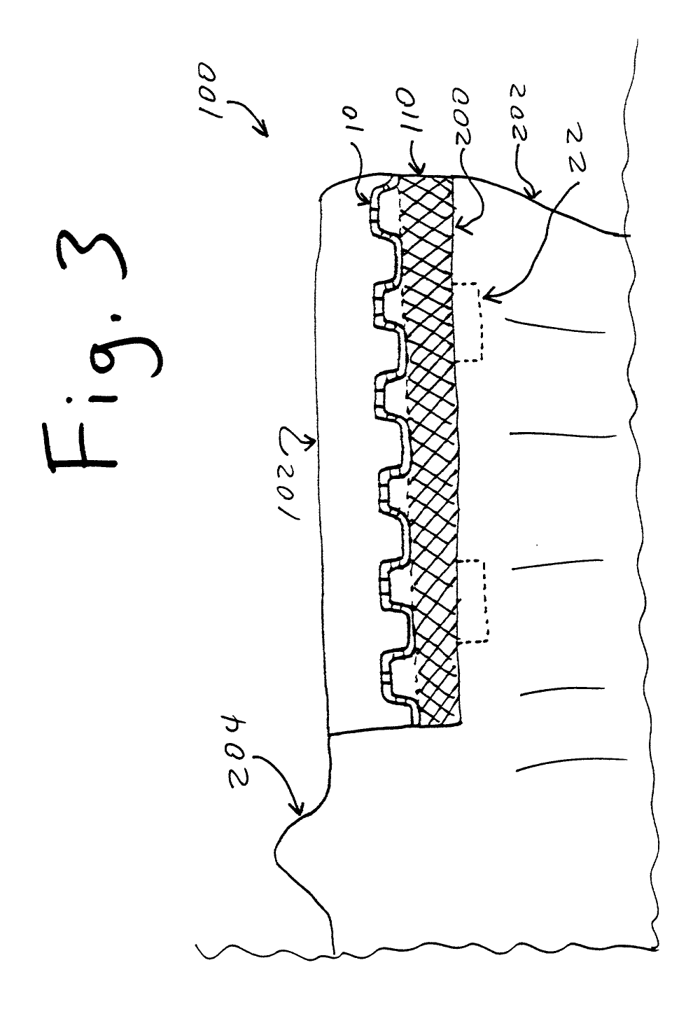

Referring to the drawings, callout number 10 in FIG. 1 refers to a “multi-perforated non-planar” (MP/NP) interface layer, designed for use as a reinforcing component inside a cartilage replacement implant. A complete implant 100 is shown (in a side cutaway view) in FIG. 3, having hydrogel layer 102 on its articulating surface, MP/NP interface layer 10 in the middle, and an anchoring layer 110 that rests against a prepared surface 200 of tibial bone 202.

The MP/NP layer 10 as shown in FIGS. 1 and 3 is sized and shaped in a manner that will render it suitable for implantation on top of a “tibial plateau” (i.e., the upper surface on top of a tibia, also called a shinbone, inside a knee joint). This type of implant is designed for a “unicompartmental” repair, which involves replacing damaged cartilage in either a medial compartment or a lateral compartment, but not both. As is well known, the tibial plateau has two roughly parallel compartments, each of which is covered by cartilage. These are designated as the “medial” (inside) and “lateral” (outside) compartments. They are separated by a raised “tibial spine” (shown as 204, in FIG. 3) in the middle of the tibial plateau; the tibial spine projections are not covered with cartilage.

In a complete implant device, the upper surface of MP/NP layer 10 will be covered by a layer of hydrogel, a hydrophilic polymer, or a similar material which is relatively soft, and which has a surface that will remain wet and lubricated by synovial fluid, inside a mammalian joint. The hydrogel or polymer will provide a smooth and lubricious “articulating” surface, which will press and slide against another cartilage segment (or another implant) within the joint that is being repaired (e.g., against a femoral runner, to continue the example of an implant for a tibial plateau).

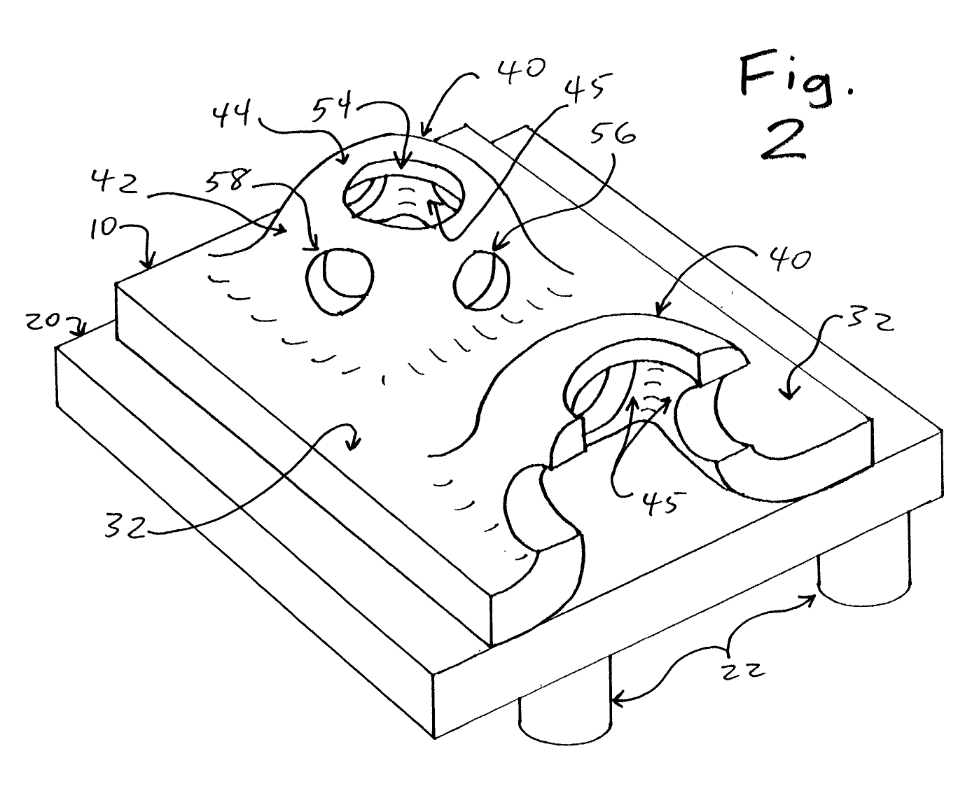

In one preferred embodiment, the lower side of MP/NP layer 10 can be bonded (by a strong and permanent adhesive, or other suitable means) to a hardened shell or tough membrane, which can be shaped, if desired, so that it will act in a manner comparable to a shallow tray, with short side walls that can help provide lateral support to both the MP/NP layer 10, which is nestled securely in the tray, and to the hydrogel or soft polymer layer which rests on top of the MP/NP layer 10. This type of shell or tray, which is shown in cross-section as bottom layer 20 in FIG. 2, can be anchored directly to a prepared bone surface, using pins and/or cement, or any other suitable means.

If desired, the anchoring layer can also be provided with downward-projecting “pegs”, such as pegs 22 as shown in FIGS. 2 and 3. A plurality of such pegs, on the bottom of an implant, can fit into accommodating holes or slots that are cut, drilled, or grinded into a bone surface while it is being prepared to receive an implant. These pegs can increase the ability of an implant to withstand shear stresses, which can be especially important for implants in joints such as knees or hips. Such pegs may be made of any suitable material, which may include: (i) impermeable polymers, such as the same polymer used to make an impermeable shell or tray 20; (ii) a biocompatible wire mesh, which can be sputter-coated if desired with a calcium-phosphate compound that emulates the hydroxyapatite crystalline structure of bone; and, (iii) a stiff and hard material, made from the same polymeric, proteinaceous, or other fibers that are present in the hydrogel, but with a much lower moisture content (e.g., less than about 10% water content, as distinct from more than 90% water content for most hydrogels).

In an alternate preferred embodiment, shown in a side cutaway view in FIG. 3, rather than using an impermeable shell/tray component 20, MP/NP layer 10 can be bonded to a porous anchoring layer 110, which can be made of a porous layer that will promote cell and tissue ingrowth into the porous layer. Porous devices made of biocompatible materials that promote tissue ingrowth are well-known in surgical implants, and can establish very strong and secure bonding with surrounding tissue. Candidate materials for such a layer include wire mesh (coated if desired), fibrous matrix, open-cell foam, etc. If desired, anchoring layer 110 can be made of the same types of polymeric, proteinaceous, or other fibers that are present in the hydrogel layer 102, but with a much lower moisture content in the stronger and stiffer anchoring layer (e.g., less than about 10% water content, as distinct from more than 90% water content for most hydrogels).

In another potential embodiment, it may be possible to dispense with a separate and distinct anchoring layer 20 or 110, and bond the relatively flat and planar underside of grid 30 (discussed below), directly to a prepared bone surface, using pins and/or cement. In this potential embodiment, the MP/NP interface layer would still provide reinforcement at the interface between a soft material (i.e., a gel or soft polymer layer, with an articulating surface) and a hard material (i.e., the prepared bone surface).

In various preferred embodiments, an implant that contains an MP/NP layer can be permanent and non-resorbable, and intended to remain inside the repaired joint for the remaining life of the patient. In various alternate preferred embodiments, all or a portion of such an implant can be designed to be gradually resorbed and replaced by naturally generated tissue.

The anchoring layer of a tibial implant device must be securely anchored (preferably using pins and/or cement) to a hard surface on a tibial bone, such as prepared surface 200 on tibial bone 202, as shown in FIG. 3. Before an implant is inserted into a knee joint, this hard bone surface must be prepared by cutting, grinding, and pulling away the damaged cartilage segment, thereby exposing a hard bone surface that will provide a solid and secure anchoring surface that can permanently support the implant.

Various tools and tool guides for arthroscopically preparing that type of hard bone surface are known to orthopedic surgeons, and additional specialized tools and tool guides for that purpose are described in U.S. Pat. No. 6,132,468, invented by the same Applicant herein. The contents and teachings of that patent are incorporated by reference, as though fully set forth herein.

Accordingly, if an implant as disclosed herein is designed for arthroscopic implantation, through a small skin incision, the anchoring layer can be generally in the form of a tough but flexible membrane, which can be curled up and rolled into a cylindrical shape that can be pushed into a joint through an insertion tube having roughly the thickness of a finger.

Alternately, the type of implant devices disclosed herein may be surgically implanted by means of “open joint” surgery, if desired. If an implant is designed for implantation using “open joint” surgical techniques, the anchoring layer may be a more rigid and less flexible.

The method of implantation is not critical to this invention; instead, the important feature of this invention is that it provides an improved interface between a relatively hard material and a much softer material.

FIG. 2 shows, in greater detail, a close-up and partial cutaway view of two perforated “riser bumps” that can be used in a preferred design for the multi-perforated non-planar (MP/NP) layer 10. This interface device 10 is also illustrated, in color-coded computer-generated drawings which were submitted with the above-cited provisional application serial No. 60/250,091, by the same inventor herein, with a purple color indicating the molded material that forms the grid and riser bumps, and with the various perforations shown in yellow, green, and blue; the anchoring shell/tray 20 is also shown, in a light-gray color.

As shown in FIG. 2, the MP/NP interface 10 includes an essentially planar base or grid 30, comprising numerous relatively flat, straight, narrow segments or rows, arranged as “longitudinal” segments 32 and “transverse” segments 34. As used herein, the term “longitudinal” refers to the longest axis or dimension of an implant, while “transverse” is the direction perpendicular to longitudinal. Other terms, such as the “A-P” direction (for anterior-posterior) and the “M-L” direction (for medial-lateral) can also be used if desired, if the intended orientation of an implant with respect to the limbs or joints of an intended recipient is known.

The grid segments 32 and 34 separate and determine the spacing of riser bumps 40, which are sloping and elevated segments or protrusions that rise substantially above the level of grid 32. Riser bumps 40 should be tall enough (i.e., above the dominant plane of grid 32) to exert substantial resistance to fluid pressures and shear stresses within a hydrogel that has completely filled and saturated both (i) the rows or aisles between riser bumps 40, and (ii) the volumes within riser bumps 40. Those two different zones, both of which should be completely filled with a gel or other soft material in a complete cartilage-replacement implant, will be in fluid communication with each other, via a large number of perforations 54, 56, and 58, which pass through the top and side surfaces of the riser bumps 40.

For maximal strength, a “large plurality” of riser bumps should be provided, in an MP/NP layer as disclosed herein. As used herein, the term “large plurality” indicates that at least a dozen or more (and preferably several dozen) riser bumps should be provided, within a device that is large enough to serve as, for example, a tibial plateau or femoral runner replacement. At the current time, it is anticipated that, for implants which use hydrogels, elevations of roughly 3 to roughly 15 mm are in the preferred range for riser bumps, and widths in the range of 5 to about 20 mm are in the preferred range; these dimensions preferably should be tested and optimized, based on the ability of such hydrogel implants to withstand imposed compressive and shear loads without suffering tearing or other damage. In addition, it should be noted that: (i) preferred dimensions for riser bumps in devices which use polymers, foams, or other materials may be substantially different; and, (ii) preferred dimensions for riser bumps will also depend heavily on the intended total thickness of an implant, including its hydrogel layer.

In the embodiment shown in FIGS. 1 and 2, riser bumps 40 are arranged in a rectangular geometric pattern, in a manner referred to herein as a “waffle” pattern or surface. In this configuration, the “longitudinal” grid segments In alternate preferred embodiments, other geometric configurations can be used, such as: (i) a diagonal or diamond array (i.e., having slanted tetrahedrons, either with or without perpendicular corners); or, (ii) a hexagonal array, often referred to as a honeycomb, with riser bumps having circular or hexagonal shapes when viewed from above. These types of geometric configurations are referred to herein as “arrays”.

As shown more clearly in the close-up cutaway view provided in FIG. 2, each riser bump 40 has one or more semi-vertical sides or facets 42, rising out of the grid 32, and a relatively horizontal upper surface 44 (all references herein to directions such as vertical or horizontal assume that the grid 30 is horizontal). If square or rectangular protrusions are used, each protrusion 40 will have four distinct or semi-distinct semi-vertical sides 42. If desired, these vertical sides can be classified as longitudinal or transverse facets.

The semi-vertical sides 42 preferably should be provided with rounded corners, if any corners are present, to minimize any risk of internal cutting, abrasion, or similar damage (such as, for example, if a patient with one of these devices in a knee or hip joint falls, or must jump down from an elevated height, and the knee or hip undergoes an instant of high compression during impact). For similar reasons, any riser bump 40 should have a flat horizontal upper surface 44, to minimize any risk that a sharp or spiked surface might be pushed into or through the soft material which will overlay it.

If desired, some or all of the riser bumps can be in the shape of rounded domes or semi-circles (which may include spherical, ellipsoid, cycloid, or similarly-shaped domes). Alternately, other desired non-planar surface shapes can be used (such as rippled or undulating surfaces, mushroom-shaped or similar structures with enlarged heads, etc.), so long as the selected shape provides an interface structure that meets the goals and requirements set forth herein.

As shown in FIG. 2, each roughly square protrusion 40 provides five facets (four roughly vertical facets 42, and a horizontal facet 44). Each of these five facets has a hole (which can also be called a perforation, orifice, aperture, etc.) passing through it, shown in FIG. 2 as horizontal holes 54, longitudinal holes 56, and transverse holes 58.

Acting together, riser bumps 40 and the numerous perforations 54-58 which pass through the riser bumps in various directions create a complex non-planar perforated outer surface, in MP/NP layer 10. In addition, as can be seen from the visible “inner walls” or “underside walls” 45, shown in FIG. 2, the outer-surface facets 42 and 44 are further supplemented by still more surfaces or facets, on the underside of the MP/NP layer 10, and on the partially-covered surface of an anchoring layer 20 or 110.

All of those surfaces or facets are exposed and accessible to the water molecules in a gel compound. Therefore, all of those surfaces or facets can resist fluid pressure which is imposed on those facets. In this manner, the complex surface geometry of this type of implant can allow this type of implant to use fluid flow, within a hydrogel, to redistribute and disseminate, in a more balanced, even, and reinforced manner, the compressive and/or shear forces that are imposed on the articulating surface 102 of an implant 100.

After implant 100 has been fabricated for use in surgically creating a cartilage replacement device, it is filled with a liquid reagent or mixture that will subsequently undergo a “setting” reaction. This type of reaction is also referred to by various other terms, such as curing, hardening, coagulating, etc., and possibly by terms such as polymerizing, crosslinking, etc., depending on the type of chemical reaction(s) involved.

The most common result of that type of reaction, in the context that is of interest herein (i.e., which involves improved interfaces between hard materials and soft materials) is to convert the liquid reagent or mixture into one of several types of relatively soft and pliable material, such as a gel. As used herein, the term “gel” implies an aqueous or “hydrogel” material; i.e., water is used to provide the small and mobile molecules (often called the solvent, fluid, or hydration molecules) which will permeate throughout the crosslinked fibrous network which holds the gel together, when the gel is in its wet and flexible (“hydrated”) form. For various reasons, hydrogels are of great interest in medicine, and are generally preferred whenever body fluids will directly contact a substance that is not covered by a water-tight membrane or other surface.

However, the current invention is not limited to use with hydrogels. It is also anticipated to be useful for various other uses wherein a relatively soft and pliable material must be securely bonded to a surface of a substantially more rigid material. Such soft and vulnerable materials include, by way of example: (1) “gel-like” materials; this term is broad enough to include gels made of synthetic polymers or similar substances which do not use water as the solvent/hydration compound; and (2) non-rigid polymers and copolymers, including but not limited to open-cell and closed-cell foams and similar compounds, which are relatively soft and vulnerable to tearing and damage if subjected to shear forces.

In a typical manufacturing operation, a gel-forming reagent or mixture is loaded, in liquid form, into a device as disclosed herein, by means such as pouring or injection. Depending on the viscosity and other traits of the liquid, this filling step may be carried out with the aid of additional forces or conditions (such as elevated temperature and/or pressure, centrifugal force, rocking, vibrating, or jarring motion, etc.) to ensure that the liquid will permeate into and throughout all of the interstitial spaces within the device.

Some type of setting reaction will then be carried out, to convert the liquid into a gel or comparable soft material. The details of such steps are known to those skilled in the art, and will depend on the type of liquid reagent or mixture that was used. For example, if a polyvinyl alcohol compound or mixture is used, an interface-and-anchor device as disclosed herein may be subjected to a cyclic freeze-thawing operation. Alternately, if a polymerizing, crosslinking, or similar chemical reaction is involved, the setting procedure may involve maintaining the liquid-loaded device under suitable conditions (which may include heating, elevated pressure, etc.) for an appropriate period of time, to allow the reaction to proceed to completion (or possibly to a level of partial completion, before a quenching step is used to terminate the reaction and prevent the material from moving beyond a desired pliable state and into a more rigid, less supple form).

After the setting reaction is complete, any appropriate finishing steps may be carried out, depending on the type of gel or other soft material that is involved, and the design goals of the final device. For example, if a surplus of gel-setting material was loaded into a shell structure, the solidified material may be carved, sculpted, trimmed, glazed, irradiated, chemically treated (such as by a crosslinking agent that will penetrate into the shallow surface layers of the soft material), or otherwise manipulated in a manner which completes and finishes the outer surface into a desired final shape having desired surface traits (including smoothness, toughness, resistance to tearing, etc.). Numerous types of finishing options are known to those skilled in the art, and may be selected and used as appropriate. In most cases, any surface-finishing steps will not be affected by the presence of a MP/NP interface device disclosed herein, which in most cases will remain fully covered and protected, well below the exposed surface of the gel, foam, or other soft material.

If desired, after a soft and pliable material such as a gel or foam has been properly emplaced within the shell and interface supports as disclosed herein, the exposed external surface of the soft material may be covered by a selected type of membrane. An example of a membrane that may be suitable for such use is disclosed in U.S. patent application Ser. No. 09/393,522, filed by the same Applicant herein. The contents of that application are incorporated herein by reference, as though fully set forth herein.

Alternately or additionally, a gel, gel-like, or similar soft material used in a device as disclosed herein can also be internally supported by a three-dimensional woven structure, such as disclosed in provisional patent application 60/192,482, filed by the same Applicant herein. The contents of that application are incorporated herein by reference, as though fully set forth herein.

Thus, there has been shown and described a new and useful means for creating a stronger and more secure interface between a gel-like or other soft material, and a substantially harder material such as an anchoring membrane or shell. Although this invention has been exemplified for purposes of illustration and description by reference to certain specific embodiments, it will be apparent to those skilled in the art that various modifications, alterations, and equivalents of the illustrated examples are possible.

1. A device for reinforcing an interface between a soft material and a hard material, comprising a non-planar layer having multiple perforations, wherein the non-planar layer comprises a relatively planar base grid having a large plurality of elevated segments distributed across the base grid and spaced apart from each other and rising above the base grid, each elevated segment having an upper surface and a lower surface, wherein:
(i) the elevated segments establish a large plurality of semi-vertical facets on the upper and lower surfaces of the elevated segments; and
(ii) the semi-vertical facets on the elevated segments are perforated in a plurality of directions, thereby allowing fluid communication between the upper and lower surfaces of each elevated segment.

2. The device of claim 1, having an array of elevated segments substantially as shown in FIG. 2.

3. The device of claim 1, wherein the elevated segments are arrayed in a geometric orientation selected from the group consisting of rectangular, diagonal, and honeycomb arrays.

4. The device of claim 1, wherein the non-planar layer is in fluid communication with a hydrogel material, and the device is intended for surgical implantation to repair damaged cartilage in a mammalian joint.

5. A surgical implant for replacing damaged cartilage in a mammalian joint, containing a non-planar layer having a large plurality of semi-vertical facets and a large plurality of perforations, positioned beneath a hydrogel layer which provides an articulating surface for the surgical implant,
wherein the semi-vertical facets and perforations interact to allow water molecules in the hydrogel to redistribute fluid pressure against the semi-vertical facets, thereby reinforcing the hydrogel layer in a manner which allows the hydrogel layer to withstand greater shear stresses without damage.

6. The surgical implant of claim 5, having an array of semi-vertical facets and perforations substantially as shown in FIG. 2.

7. The surgical implant of claim 5, wherein the semi-vertical facets are arrayed in a geometric orientation selected from the group consisting of rectangular, diagonal, and honeycomb arrays.
